(19) United States
(12) Patent Application Publication
(10) Pub. No.: US 2007/0016155 A1
(43) Pub. Date: Jan. 18, 2007

(54) Shaped side panel for disposable absorbent article

(76) Inventors: Kuo-Shu Edward Chang, Charlotte, NC (US); Andrew Wright, Chesterfield (GB); Patrick King Yu Tsang, Matlock (GB); Steven D. Linton, Snellville, GA (US)

Correspondence Address: John F. Klos;Fulbright & Jaworski LLP, Suite 2100, 80 S. Eighth Street, Minneapolis, MN, 55402, US

(21) Appl. No.: 11/474,653

(22) Filed: Jun. 26, 2006

Related U.S. Application Data
(63) Continuation-in-part of application No. 10/733,649, filed on Dec. 11, 2003.

(51) Int. Cl. A61F 13/15

(52) U.S. Cl. 604/385.3; 604/389

(57) ABSTRACT

A disposable absorbent article defining an absorbent chassis adapted to wrap around portions of a torso and a pair of side panels adapted to secure the absorbent chassis about the torso during use. The side panels include at least a pair of substantially inextensible zones and a zone of substantial elasticity providing partial extensiblity to the side panels. The side panels may be secured to the chassis at one of the inextensible zones and be provided with a fastener component at another inextensible zone. At least portions of opposed edges of the side panels are shaped into a nonparallel configuration to improve fit, comfort and exudate control of the absorbent article.

### RELATED APPLICATION

This application is a continuation-in-part of U.S. application Ser. No. 10/733,649, filed Dec. 11, 2003.

### TECHNICAL FIELD

The present invention relates generally to disposable absorbent articles and more particularly to waste worn absorbent articles.

### BACKGROUND OF THE INVENTION

Absorbent articles, such as diapers, training pants and incontinence garments, provide a close, comfortable fit about the wearer and contain body exudates when such articles perform properly. Conventional absorbent articles include a front waist portion and a rear waist portion, which are releasably connected about the hips of the wearer during use by fasteners, such as adhesive tape fasteners or hook and loop type fasteners attached to a side panel, such as an elasticized ear, flap, tab or enlarged panel. For example, a conventional fastening system may have a pair of fasteners, such as adhesive tape tabs, located upon an ear or side flap structure, and a complimentary fastener, such as a taping panel, located on the outer surface of the outer cover of the diaper in the front waist portion of the diaper. In a diaper configuration, the absorbent article can be positioned between the legs of the wearer while the wearer is lying down and the adhesive tape tabs then releasably attached to the taping panel to secure the rear waist portion to the front waist portion of the diaper to secure the diaper about the waist of the wearer. In a training pant configuration, the side panels may be substantially larger than typical ears and with panel portions extending, for example, from a waist edge to a leg engaging edge.

Prior art absorbent articles having side panels and conventional fastening systems have not been completely satisfactory and user needs of comfort, fit and exudate control remain elusive goals. Typical side panels are relatively narrow with generally parallel opposed side edges. Such side panels having generally linear edges can cause skin irritation and/or injury to the user's skin, as the linear edges have a tendency to focus garment retention forces to relatively narrow regions of the user's skin. Additionally, the forces exerted on the fastening system are often inefficiently transferred and distributed across the waist region of the diaper. As a result, the fit, comfort and exudate containment of conventional absorbent articles have not been completely satisfactory.

### SUMMARY OF THE INVENTION

A disposable absorbent article providing better fit, comfort and exudate control through application of an improved side panel structure. The side panel structure may be utilized, for example, on disposable absorbent articles such as diapers, disposable pull-on garments, and the like. Disposable pull-on garments include training pants, pull-on diapers, disposable underwear, and adult incontinence garments.

Side panel structures in accordance with the present invention may be embodied as ears, tabs or larger panels extending laterally from side edges of an absorbent chassis near the front waist region, the rear waist region or both.

Side panel structures in accordance with the present invention address many limitations of the prior art. Advantages provided by contoured or “shaped” panels side panels of the present invention include improved garment fit and comfort. Another advantage of such side panels is the minimization of user skin injury as garment attachment forces are transferred across a relatively wide, contoured side panel.

A side panel structure in accordance with the invention includes one or more elastic zones providing panel extensibility and a plurality of inelastic zones. Each elastic zone may include one or more elastic members, such as threads, strands, ribbons, nets and films.

A side panel structure in accordance with the invention is provided in a “shaped” configuration, with a free end, an opposite end attached to the absorbent chassis, and a pair of opposing side edges extending between the ends. At least portions of the opposing edges sides are shaped in substantially nonparallel configuration. Shaped side panels in accordance with the invention may embody opposing side edges having, for example, tapered or concave portions extending across one or more inextensible zones and/or elastic zones.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a plan view of a disposable absorbent article in the unfolded configuration.

FIG. 2 is a plan view of a side panel of FIG. 1.

FIG. 3 is a perspective view of the side panel of FIG. 2 with a cut-out detail to show an elastic construction.

FIG. 4 is a plan view of a disposable absorbent garment having large “non-shaped” side panels.

FIG. 5 is a plan view of another alternative disposable absorbent garment having “shaped” side panels.

FIG. 6 is a plan view of an alternative side panel according to the present invention.

FIGS. 7-9 are plan view of alternative disposable absorbent garments having shaped side panels.

FIGS. 10-20 are partial plan views of alternative disposable absorbent articles having shaped side panels in accordance with the invention.

### DETAILED DESCRIPTION OF THE INVENTION

For purposes of the present description, the term “side panel” is used to refer to a structure that extends laterally away from an absorbent chassis at a rear region, front region or both and being joined to the opposing waist region to allow for fitting of the absorbent article about the waist of the user. Side panels may be embodied, for example, as ear portions, tabs or larger panels. Side panels may be secured to an absorbent chassis at a side margin or may be integrated within the overall absorbent article design and/or construction. Side panels may include multiple plies of material along with one or more elastic elements such as, but not limited to, elastic threads, strands, ribbons, nets and films.

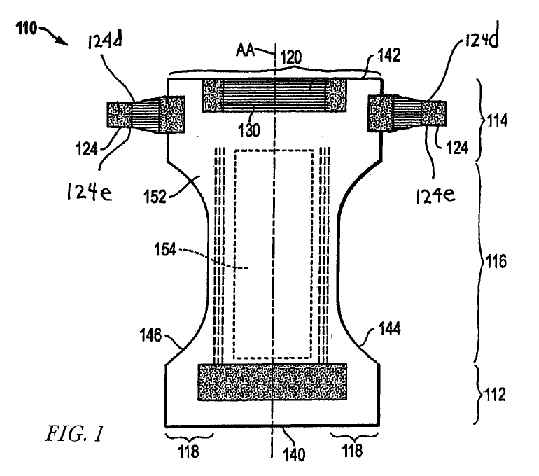

In FIG. 1, an embodiment of an absorbent article in accordance with the invention is shown as garment 110 having a pair of shaped side panels 124. Disposable absorbent garment 110 is of a type that can be placed against or in proximity to the body of a wearer so as to absorb and to contain various bodily exudates. It should be noted, however, that the present invention is applicable to a variety of disposable absorbent articles and garments, including training pants and a variety of adult incontinence products.

Garment 110 includes an absorbent chassis which generally defines three main regions aligned along an imaginary longitudinal axis or plane AA. These regions include a first waist region 112 (typically at the front of the user when the garment 110 is worn), a back waist region 114, and a crotch region 116. Diaper 110 is also characterized by a front edge 140, a back longitudinal edge 142, a first lateral or side edge or side margin 144, and a second lateral or side edge or side margin 146. In this embodiment, the absorbent chassis is formed by a liquid permeable inner layer or topsheet 152, a liquid impermeable outer layer or backsheet (not shown), and an absorbent core 154 sandwiched between the two layers.

Side panels 124 are secured at or near the side margins 144, 146. Side panels 124 are used to attach the waist regions 112, 114 together to secure garment 110 to a user. In this embodiment, side panels 124 are shaped to include a pair of nonparallel opposite side edges 124d and 124e. As illustrated, portions of side edges 124d and 124e taper inwardly toward each other. In other embodiments of the invention, portions of side edges 124 may taper outwardly, that is, the side edges may expand in relation to the distance from the absorbent chassis. A side edge 124 having an outwardly tapering configuration could be secured at one end to the front waist region of the garment 110, with the free (wider) end being refastenably connected to the rear waist region during use.

Garment 110 also has an elastic waistband 130 positioned in this embodiment generally along the back edge 142 to facilitate fastening and to enhance the fit and seal of the garment 110. When the hourglass-shaped garment 110 is worn, the crotch region 116 fits about the crotch of the wearer, and the front and back waist regions, 112 and 114, fit about the corresponding waist areas. The shaped side panels 124, wrap at least partially about the wearer to secure garment 110 during use. In other embodiments of garment 110, waistband 130 may be provided along the front edge 140, rear edge 142 or both.

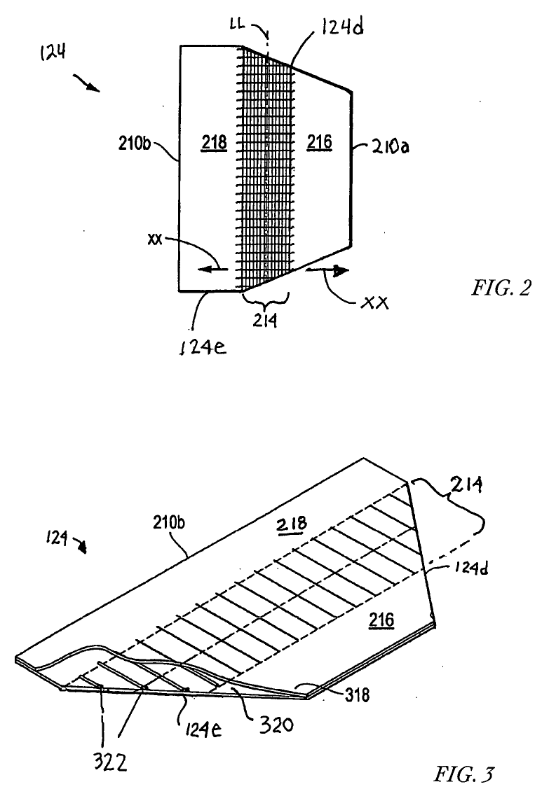

FIG. 2 depicts shaped side panel 124 removed from the absorbent chassis. Shaped side panel 124 has a central zone 214 in which an elastic construction is situated. Extending laterally from this central elastic or elasticized zone 214 are zones 216 and 218, which are substantially non-elasticized. Inelastic zone 216 provides a working area on which fastening materials and other accessories or structural attributes of the disposable absorbent garment may be situated. For example, a component of a hook and loop fastening system and/or an adhesive element may be situated within inelastic zone 216. Inelastic zone 218 may provide an attachment zone within which side panel 124 is secured to the absorbent chassis.

Shaped side panel 124 has end edges 210a and 210b and side edges 124d and 124e. Side edges 124d, 124e include portions which are parallel (along inelastic zone 218) and portions which converge (along zone 214 and inelastic zone 216). Side panel 124 stretches, in the lateral direction (denoted by arrows XX).

FIG. 3 provides a perspective view and partial cut-out of shaped side panel 124. Shaped side panel 124 has a top layer 318 and a bottom or base layer 320. The two layers 318, 320 preferably extend the total width and length of side panel 124. Both base layer 320 and top layer 318 are preferably a breathable, disposable material such as propylene fabric, breathable polyethylene/polypropylene films, or non-porous films (or combinations of these materials). Base layer 320 and/or tope layer 318 may include woven and/or non-woven fabrics. Base layer 320 and top layer 318 adhere to one another, thereby sandwiching and securing a plurality of elastic elements 322 therebetween. Elastic elements 322 may include, for example, one or more of threads, ribbons, mesh, apertured film and other films. Thread-form elastic elements 322, as shown in FIG. 3, may be substituted, in alternative embodiments, by suitable elastic elements such as elastic strands of different construction, nets, threads, ribbons, and elastic glue beads.

The elasticized zone 214 of side panels 124 may be implemented in a variety of ways. For example, an elastic zone 214 may contain a single piece of stretchable elastic material or a combination of individual pieces or panels of elastic material. The elastic zone or at the least individual pieces or panels of the elastic zone may comprise elastic composite materials or non-stretchable materials which have been rendered elastically contractible by means known to those skilled in the art. In some embodiments, the elastic zone includes a two-ply stretch panel comprising a top layer, a bottom layer and a plurality of intermediate elastic ribbons. In another configuration, the elastic zone may be provided by a substantially non-elastomeric material, such as polymer films, woven fabrics, non-woven fabrics, or the like such as described above as being suitable for the outer cover or bodyside liner. Such an elastic zone could be modified to render portions elastically contractible to provide the desired elastic properties. In another example, the elastic material may be latent, in which case the elastic material would initially have non-elastomeric properties, but would later be activated to impart elastomeric properties to the stretch panel by any of various means known to those skilled in the art. Additional details of non-shaped side panel construction, including embodiments using aligned elastic threads, are disclosed in U.S. Ser. No. 10/733,649, and U.S. Ser. No. 11/113,114, and each application being incorporated by reference herein.

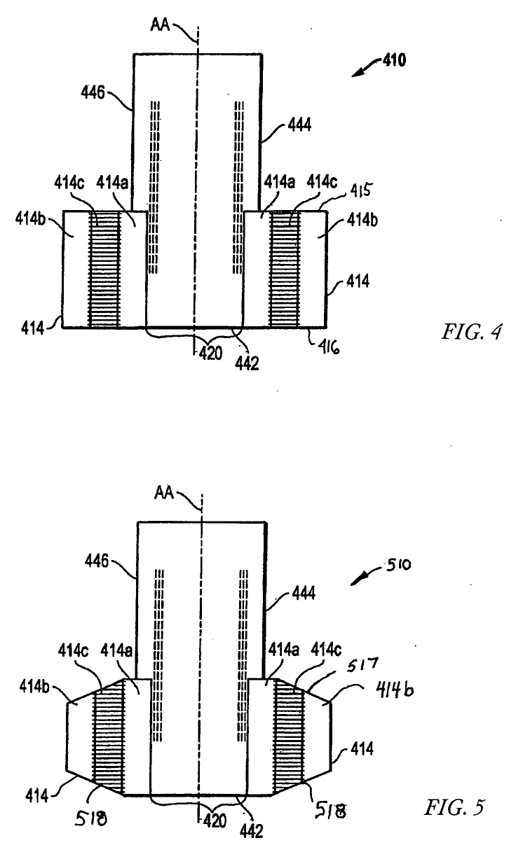

FIG. 4, provided for comparative purposes only, depicts a disposable absorbent garment 410 having a pair of “non-shaped” side panels 414. Side panels 414 are separately attached to a central body 420 of garment 410. Side panels 414 include side edges 415, 416 that are generally parallel, with side edge 416 defining at least a portion of a waist edge 442 of garment 410 and side edge 415 defining at least a portion of a leg edge. Side panels 414 have a non-elasticized zone 414b that is positioned outboard of the side margins 444, 446 of the garment 410 and a second non-elasticized zone 414a, at least a portion of which is attached inboard of the side margin 444, 446. Thus, a central elastic zone 414c is situated outboard of the side margin 444, 446 and not directly attached thereto. When garment 410 is worn, the central elasticized zone 414a allows the side panel to stretch in a lateral direction. Accordingly, during garment use side panels 414 allow for stretching about the waistline of the user. In another embodiment of the invention, another pair of side panels 414 may be secured near the front waist region providing a garment 410 with 4 side panels 414.

FIG. 5 depicts an alternative disposable absorbent garment 510 according to the invention. Certain aspects of garment 510 are numbered similarly to corresponding aspects of garment 410. In comparison to the side edges 415, 416 of FIG. 4, garment 510 includes side edges 517, 518 which are substantially nonparallel across portion of side panels 414. For example, portions of side edges 517, 518 within elastic zone 414c and inelastic zone 414b are generally tapered resulting in a reduction of side panel 414 width in a transverse direction of garment 510. While a side panel 414 with generally linear taper is shown in FIG. 5, other side panel 414 shapes may include curves or combinations of lines and curves. For example, FIGS. 8, 12, 13, 15 and 17 illustrate side panels with portions of side edges extending in nonlinear manners.

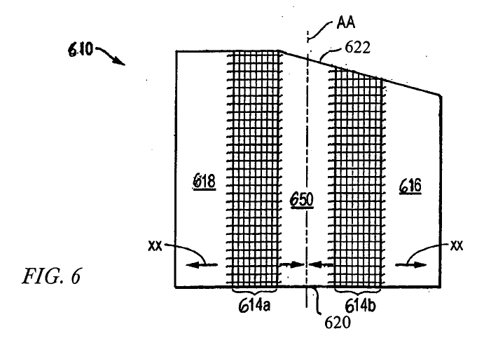

FIG. 6 depicts an alternative embodiment of a side panel 610 suitable for use with embodiments according to the present invention. Side panel 610 illustrated therein differs from the previously described side panels (124, 210, 414) in that side panel 610 includes two elasticized zones 614a and 614b. Elasticized zones 614a, 614b may be equidistantly spaced apart on either side of the longitudinal centerline AA. The spacing of the elasticized regions 614a, 614b creates right and left non-elasticized or dead zones 616, 618, as well as central non-elasticized region 650. The elasticized regions 614a, 614b imparts elasticity to side panel 610 in the lateral directions XX. Side panel 610 includes side edge 620 which extends in a linear manner across inelastic zones 618, 650, 616 and elastic zones 614a, 614b, and another side edge 622 which extends in a parallel manner to side 620 within zones 618 and 614a, and extends toward side edge 620 in a tapering manner within zones 650, 614b and 616.

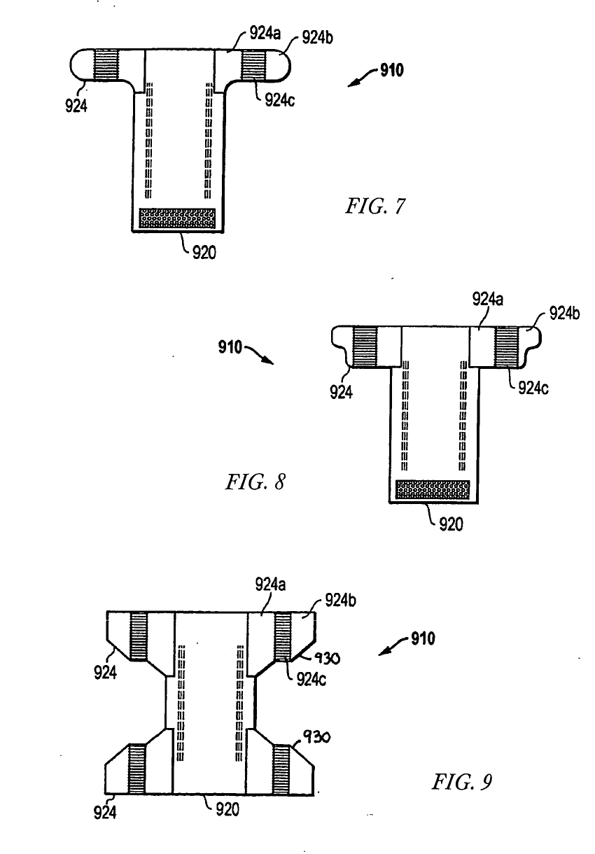

Referring to FIGS. 7 and 8, a disposable absorbent garment 910 is shown having a central body 920 and ear- and tab-shaped side panels 924 positioned proximate to a waist edge. Side panels 924 have inner and outer nonelasticized zones 924a, 924b, and a central elasticized zone 924c situated therebetween. FIGS. 7 and 8 illustrate side panels 924 having shaped nonelasticized regions 924a and 924b, as, for example, portions of side edges of side panels 924 converge within the inelastic zone(s) 924a, 924b. In both designs, portions of the side edges of the outer nonelasticized regions 924b are rounded or curved.

Now turning to FIG. 9, yet another variation of side panel 924 is shown applied to a training pant 910. In this embodiment, side panel 924 is substantially larger than ear-shaped side panel 924 of FIGS. 7 and 8, and includes a waist edge and a leg engaging edge 930. Side panel 924 is defined by nonelasticized zones 924a and 924b having different geometries.

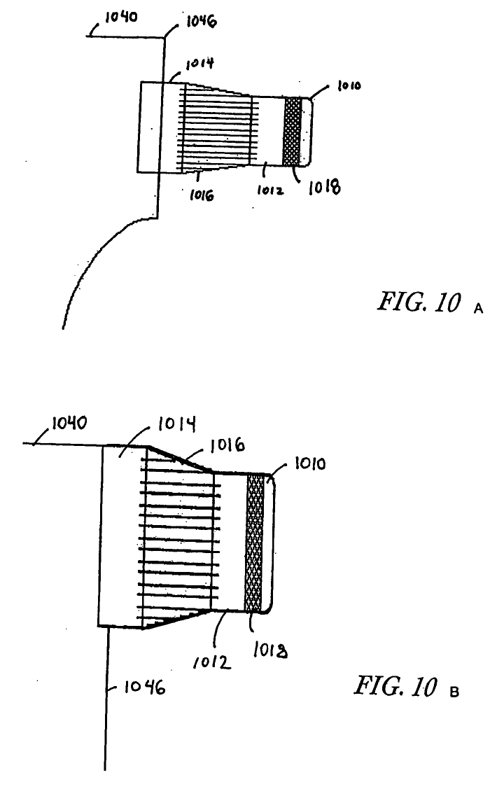

FIGS. 10-20 illustrate further aspects of the present invention. In each example, a disposable absorbent garment is shown having a central body and side panels in the form of ears, tabs or even larger panels. While these illustrated embodiments have side panels with inner and outer nonelasticized regions and a central elasticized region situated therebetween, other side panel embodiments may have a plurality of both elasticized and nonelasticized regions, configured for example as shown in FIG. 6.

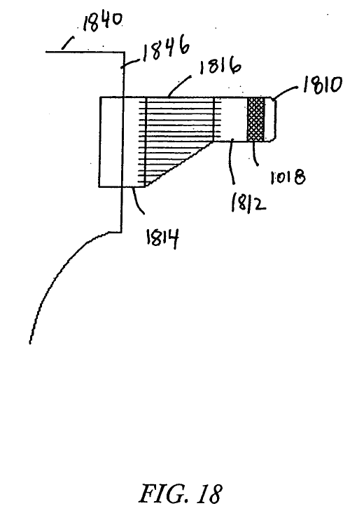

The exemplary embodiments of FIGS. 10-20 illustrate a variety of differently configured side panels, in ear, tab or even larger form. FIGS. 10-17 represent side panels which are generally symmetrical about an axis perpendicular to a longitudinal axis of the absorbent chassis. Opposite side edges of side panels of these embodiments would align if folded about the center axis. In comparison, FIGS. 18-20 represent side panels which are asymmetric about a center axis.

Turning to FIG. 10a, the side panel 1010 is shown applied at a side margin 1046 of a disposable absorbent article near waist edge 1040. Side panel 1010 has nonelasticized regions 1012, 1014 of different geometries. In the embodiment of FIG. 10, the nonelasticized regions 1012, 1014 have side edges which are generally parallel, while the side edges of elasticized region 1016 taper inwardly toward outer nonelasticized region 1012. A fastening component 1018, such as a hook and loop component is affixed or defined within nonelasticized region 1012. Fastening component 1018 may extend generally between the side edges of the side panel, or alternatively, may extend within only a portion of nonelasticized region 1012.

FIG. 10a illustrates a side panel 1010 in a “tab” configuration, while FIG. 10b shows a side panel 1010 with a substantially larger area extending generally from the waist edge to a leg edge and adapted to engage a user's leg during use.

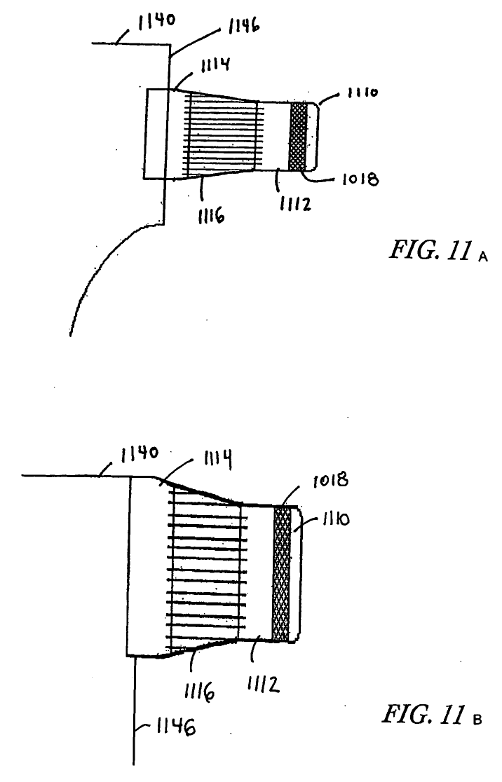

Now turning to FIG. 11, a side panel 1110 is shown applied at a side margin 1146 of a disposable absorbent article near waist edge 1140. Side panel 1110 has nonelasticized regions 1112, 1114 of different geometries. In the embodiment of FIG. 11, the outer nonelasticized regions 1112 as well as in inward portion of nonelasticized region 1114 have side edges which are generally parallel, while the side edges of elasticized region 1116 and an outer portion of nonelasticized region 1114 taper inwardly toward outer nonelasticized region 1112.

FIG. 11a illustrates a side panel 1010 in an “ear” or “tab” configuration, while FIG. 11b shows a side panel 1110 with a substantially larger area extending generally from the waist edge and having a leg edge adapted to engage a user's leg during use.

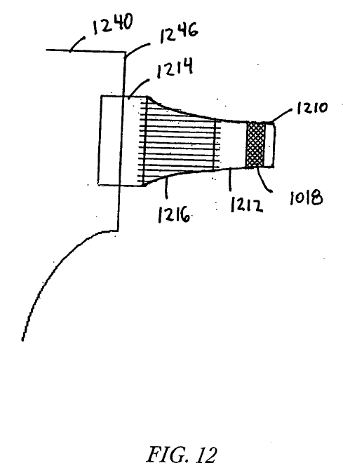

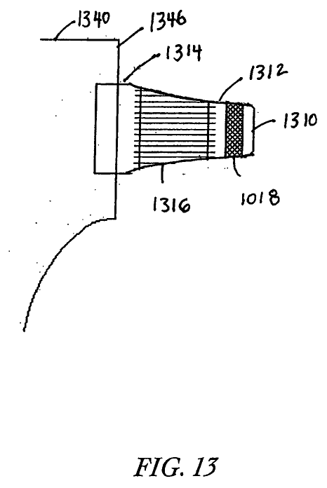

Now turning to FIG. 12, a side panel 1210 is shown applied at a side margin 1246 of a disposable absorbent article near waist edge 1240. Side panel 1210 has nonelasticized regions 1212, 1214 of different geometries. In the embodiment of FIG. 12, the nonelasticized region 1214 has side edges which are generally parallel, while the side edges of elasticized region 1216 and nonelasticized region 1212 taper inwardly. Similar to the embodiments of FIGS. 10b and 11b, side panel 1212 (and the side panels of FIGS. 13-20) can also be implemented as a substantially larger panel having, for example, a waist edge and a leg engaging edge.

Now turning to FIG. 13, a side panel 1310 is shown applied at a side margin 1346 of a disposable absorbent article near waist edge 1340. Side panel 1310 has nonelasticized regions 1312, 1314 of different geometries. In the embodiment of FIG. 13, the inward portion of nonelasticized region 1314 has side edges which are generally parallel, while the side edges of elasticized region 1316, an outer portion of nonelasticized region 1314, and nonelasticized region 1312 taper inwardly, in a concave manner.

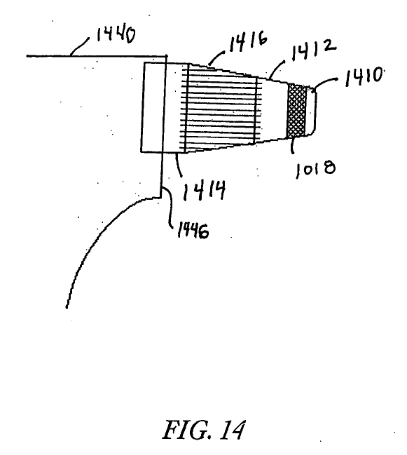

FIG. 14 illustrates a side panel 1410 applied at a side margin 1446 of a disposable absorbent article near waist edge 1440. Side panel 1410 has nonelasticized regions 1412, 1414 of different geometries. In the embodiment of FIG. 14, the inner nonelasticized region 1414 has side edges which are generally parallel, while the side edges of elasticized region 1416 and outer nonelasticized region 1414 taper inwardly. Fastening component 1418 may extend generally between the side edges of the side panel or alternatively, may extend within only a portion of nonelasticized region 1412.

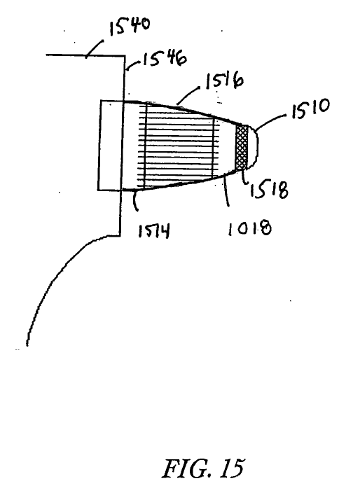

FIG. 15 illustrates a side panel 1510 as applied at a side margin 1546 of a disposable absorbent article near waist edge 1540. Side panel 1510 has nonelasticized regions 1512, 1514 of different geometries. In the embodiment of FIG. 15, the inward portion of nonelasticized region 1514 has side edges which are generally parallel, while the side edges of elasticized region 1516, an outer portion of nonelasticized region 1514, and nonelasticized region 1512 taper inwardly, in a convex manner.

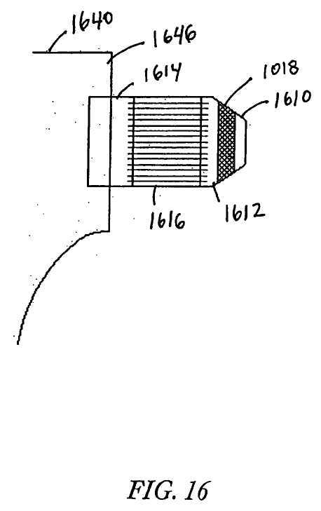

FIG. 16 shows a side panel 1610 as applied at a side margin 1646 of a disposable absorbent article near waist edge 1640. Side panel 1610 has nonelasticized regions 1612, 1614 of different geometries. In the embodiment of FIG. 16, the nonelasticized region 1614 and elasticized region 1616 have side edges which are generally parallel, while the side edges of nonelasticized region 1612 taper inwardly.

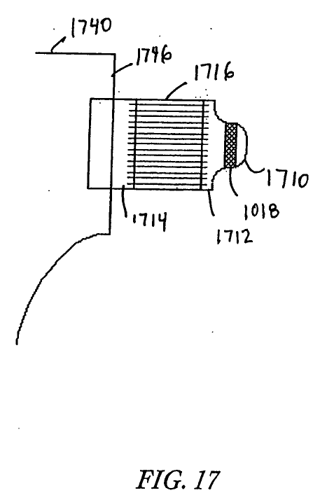

FIG. 17 shows a side panel 1710 as applied at a side margin 1746 of a disposable absorbent article near waist edge 1740. Side panel 1710 has nonelasticized regions 1712, 1714 of different geometries. In the embodiment of FIG. 17, the nonelasticized region 1714 and elasticized region 1716 have side edges which are generally parallel, while the side edges of nonelasticized region 1712 taper inwardly, in a concave manner.

FIG. 18 shows a side panel 1810 as applied at a side margin 1846 of a disposable absorbent article near waist edge 1840. Side panel 1810 has nonelasticized regions 1812, 1814. In the embodiment of FIG. 18, the nonelasticized regions 1812, 1814 have side edges which are generally parallel, while one of the side edges of elasticized region 1816 tapers inwardly.

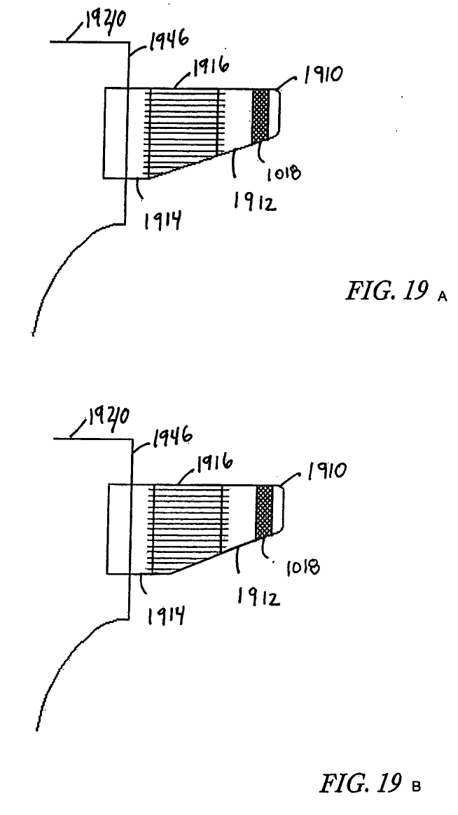

FIG. 19 shows a side panel 1910 as shown applied at a side margin 1946 of a disposable absorbent article near waist edge 1940. Side panel 1910 has nonelasticized regions 1912, 1914 of different geometries. In the embodiment of FIG. 19a, the nonelasticized region 1914 has side edges which are generally parallel, while one of the side edges of elasticized region 1816 and one of the edges of nonelasticized region 1912 taper inwardly. In comparison, FIG. 19b shows a side edges which are parallel across zone 1914 and a portion of zone 1916 and then taper inwardly across the balance of zone 1914 and zone 1912. FIG. 19b represents an elastic zone 1916 having both parallel and non-parallel side edges.

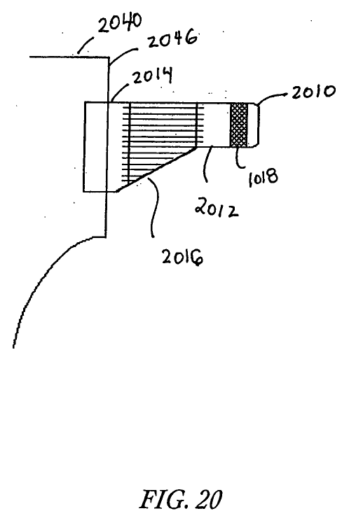

FIG. 20 shows a side panel 2010 as shown applied at a side margin 2046 of a disposable absorbent article near waist edge 2040. Elastic composite band 2010 has nonelasticized regions 2012, 2014 of different geometries. In the embodiment of FIG. 20, a portion of nonelasticized region 2014 and nonelasticized region 2012 have side edges which are generally parallel, while one of the side edges of elasticized region 2016 and another portion of nonelasticized region 2014 tapers inwardly.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A disposable absorbent article comprising:
an absorbent chassis which defines a front waist region, a rear waist region, a crotch region which extends between and connects said waist regions; and
at least one laterally extensible side panel defining a pair of inextensible zones and an intermediate elastic zone disposed between the pair of inextensible zones, said side panel being attached to the absorbent chassis at one end and being refastenably attached to the absorbent chassis at another end via a first refastenable fastener component, said side panel having a pair of opposite side edges extending between said ends, and portions of said side edges are generally nonparallel.

2. The absorbent article of claim 1 wherein the side panel is permanently attached at the rear waist region and refastenably attached to the absorbent chassis at the front waist region.

3. The absorbent article of claim 2 wherein the side panel is refastenably attached upon an outer surface of absorbent chassis.

4. The absorbent article of claim 2 wherein the side panel is refastenably attached upon an inner surface of absorbent chassis.

5. The absorbent article of claim 1 wherein the side panel is permanently attached at the front waist region and refastenably attached to the absorbent chassis at the rear waist region.

6. The absorbent article of claim 5 wherein the side panel is refastenably attached upon an outer surface of absorbent chassis.

7. The absorbent article of claim 5 wherein the side panel is refastenably attached upon an inner surface of absorbent chassis.

8. The absorbent article of claim 1 wherein the first fastener component is part of a refastenable system including a second fastener component disposed upon the absorbent chassis, and wherein the first and second fastener components cooperate to attach the side panel to the absorbent chassis.

9. The absorbent article of claim 8 wherein the second fastener component is disposed within the front waist region of the absorbent chassis.

10. The absorbent article of claim 8 wherein the second fastener component is disposed within the rear waist region of the absorbent chassis.

11. The absorbent article of claim 8 wherein the first fastener component and the second fastener component are components of a refastenable hook and loop-type fastener.

12. The absorbent article of claim 11 wherein the first fastener component is a loop-type fastener.

13. The absorbent article of claim 12 wherein the loop-type fastener is defined by a fiber surface of the side panel.

14. The absorbent article of claim 11 wherein the loop-type fastener is defined by a fiber surface of the absorbent chassis.

15. The absorbent article of claim 1 wherein the elastic zone of the side panel extends substantially between a waist edge and a leg opening edge of the side panel.

16. The absorbent article of claim 1 wherein at least one of inextensible zones extends substantially between a waist edge and a leg opening edge of the side panel.

17. The absorbent article of claim 1 wherein the first fastener component is elongated in a longitudinal direction of the diaper.

18. The absorbent article of claim 1 wherein the elastic zone and the pair of inextensible zones are comprised of generally inextensible materials.

19. The absorbent article of claim 1 wherein the side panel comprises a top ply of material and a bottom ply of material which together define portions of the elastic zone and the pair of inextensible zones.

20. The absorbent article of claim 19 wherein the elastic zone further comprises an elastic member joined to the top and bottom plies of material.

21. The absorbent article of claim 20 wherein the elastic member comprises a plurality of elastic ribbons which extend along at least a portion of the side panel generally between the pair of inextensible zones.

22. The absorbent article of claim 21 wherein an elastic ribbon is intermittently secured to an element of the side panel.

23. The absorbent article of claim 1 wherein said portions proximate to one of the pair of inextensible zones are generally parallel.

24. The absorbent article of claim 1 wherein said portions of said side edges taper inwardly or outwardly between said ends.

25. The absorbent article of claim 24 wherein said portions are within the elastic zone.

26. The absorbent article of claim 25 wherein said portions are also within at least one of the inextensible zones.

27. The absorbent article of claim 1 wherein the composite band defines m elastic zones and a m+1 inextensible zones, where m is greater than 2.

28. The absorbent article of claim 1 wherein a portion of each of the elastic zone and inextensible zones defines a leg opening edge of the absorbent article.

29. The absorbent article of claim 28 wherein the first fastener component extends generally between the waist edge and an edge of the leg opening.

30. The absorbent article of claim 1 wherein the side panels define a portion of a waist edge of the absorbent article.

31. The absorbent article of claim 1 having two pairs of said side panels, each side panel defining a portion of a waist edge and a portion of a leg edge.

32. The absorbent article of claim 1 wherein said side panel is attached to said absorbent chassis at one of the pair of inextensible zones.

33. The absorbent article of claim 32 wherein said first fastener component is at least partially located within the other one of the pair of inextensible zones.

34. An absorbent article comprising:
a chassis having an absorbent core and defining front and rear waist edges and a pair of side edges;
a pair of side panels attached to said chassis, each side panel including a plurality of zones of limited extensibility and at least one zone of substantial elasticity, and each side panel including a pair of opposed side edges extending across one or more of said zones in a nonparallel configuration; and
a fastener system for releasably securing free ends of said pair of side panels to said chassis during use.

35. The absorbent article of claim 34 wherein said side panels are attached at said side edges of said chassis within one of said plurality of zones of limited extensibility.

36. The absorbent article of claim 35 wherein one component of said fastener system is located within another one of said plurality of zones of limited extensibility.

37. The absorbent article of claim 34 wherein said opposed side edges extend across portions of said side panel in a parallel configuration and across other portions of said side panel in a converging or diverging configuration.

38. The absorbent article of claim 34 wherein said opposed side edges within said zone of substantial elasticity are converging or diverging.

39. The absorbent article of claim 34 wherein said opposed side edges are both displaced away from said waist edges.

40. The absorbent article of claim 34 wherein one of said opposed side edges is generally aligned with one of said waist edges and the other one of said opposed side edges defines a portion of a leg engaging edge adapted to wrap along a portion of a user's leg during use.

41. A packaged assembly of prefastened disposable absorbent articles comprising:
a plurality of absorbent articles each having an absorbent chassis which defines a front waist region, a rear waist region, a crotch region which extends between and connects said waist regions, and at least one laterally extensible side panel defining a pair of inextensible zones and an intermediate elastic zone disposed between the pair of inextensible zones, said side panel being attached to the absorbent chassis at one of the pair of inextensible zones and being refastenable attached to the absorbent chassis at the other of the pair of inextensible zones via a refastenable fastener component, and said side panel having opposed edges which are generally nonparallel.

42. The packaged assembly of claim 41 wherein each absorbent article includes two pairs of side panels.

43. The packaged assembly of claim 41 wherein the pair of inextensible zones and elastic zone are defined by a top layer of material and a bottom layer of material.

44. The packaged assembly of claim 43 wherein at least one of the top layer or bottom layer of material is a generally nonextensible material.

45. The packaged assembly of claim 44 wherein the elastic zone includes an elastic element connected in a pretensioned state to the nonextensible material.

46. The packaged assembly of claim 41 wherein said pair of inextensible zones and elastic zone extend substantially from one of said pair of opposed edges to the other.

47. A pant-like disposable absorbent article manufactured by a method comprising the steps of:
joining a pair of side panels to an absorbent chassis at opposed side edges thereof so that said side panels extend outwardly from said side edges, wherein each side panel defines an elastic zone interconnected between a pair of generally non-stretchable zones, wherein each said side panel is joined to the absorbent chassis and one of said pair of non-stretchable zones and other one of said pair of non-stretchable zones is provided with a refastenable fastener component, and wherein at least portions of opposite edges of said side panels are generally nonparallel.

48. The method of claim 47 wherein joining the pair of side panels includes a step of applying an adhesive bonding material to said absorbent chassis.

49. The method of claim 47 wherein joining the pair of side panels includes a step of aligning a waist edge of said side panels with a waist edge of said absorbent chassis.

50. The method of claim 47 wherein said pair of side panels are joined near a rear waist region of said absorbent article.

51. The method of claim 47 wherein said at least portions of opposite edges of said side panels include generally inwardly or outwardly tapering portions.

52. The method of claim 51 wherein said tapering portions extend in either a convex or concave manner toward free edges of said side panels.

53. The method of claim 52 wherein said tapering portions extend across portions of said pair of generally non-stretchable zones and said elastic zone.

54. The method of claim 51 wherein said tapering portions extend only across an outermost one of the pair of generally non-stretchable zones.

55. The method of claim 47 wherein said pair of side panels are generally symmetrical about an axis.
